(19) United States
(12) Patent Application Publication
(10) Pub. No.: US 2021/0358587 A1
(43) Pub. Date: Nov. 18, 2021

(54) ASSESSMENT AND RECOMMENDATION ENGINE FOR INCREASING YIELD IN A REMOTE COMPUTING ENVIRONMENT

(71) Applicant: Click Therapeutics, Inc., New York, NY (US)

(72) Inventors: Allison Hirsch, New York, NY (US); Brian Iacoviello, New York, NY (US); Caroline Pena, New York, NY (US)

(73) Assignee: Click Therapeutics, Inc., New York, NY (US)

(21) Appl. No.: 17/286,211

(22) PCT Filed: Sep. 10, 2020
(86) PCT No.: PCT/US2020/050103

§ 371 Date: Apr. 16, 2021

Related U.S. Application Data
(60) Provisional application No. 62/898,487, filed on Sep. 10, 2019.

(51) Int. Cl. G16H 20/00; G16H 50/20; G16H 40/67; H04L 29/08; G06F 3/0488

(52) U.S. Cl. CPC G16H 20/00; G16H 50/20; G06F 3/0488; H04L 67/22; G16H 40/67

(57) ABSTRACT

A computer system for assessing click speed data in a remote computing environment comprising one or more processors, one or more computer-readable memories, and one or more computer-readable storage devices, and program instructions stored on at least one of the one or more storage devices for execution by at least one of the one or more processors via at least one of the one or more memories is provided. The stored program instructions include determining a plurality of baseline click decision data (CDS) values for a user; following the determining of the plurality of baseline CDS values for the user, determining a subsequent CDS value each time a user makes a selection when prompted with a prompt; for each of the subsequent CDS values, comparing the subsequent CDS value to the plurality of baseline CDS values; determining whether the subsequent CDS value presents a predetermined deviation from the plurality of baseline CDS values; incrementing a recorded deviations for each of the predetermined deviation; if the recorded deviations exceeds a predetermined allowable number of deviations, present, on one or more screens of a user device, a warning message to the user.

### PRIORITY

The present application claims priority to U.S. Provisional Patent Application No. 62/898,487, which was filed in the United States Patent and Trademark Office on Sep. 10, 2019, the entire disclosure of which is incorporated herein by reference.

### INTRODUCTION

Personalized and artificial-intelligence based interventions allow for treatment and modification of diseases, based on feedback and response.

Such treatments were generally not possible without machine-learning and artificial-intelligence based components, which allow for iterative creation and modification of treatment plans, as well as personalization, based on real-time factors.

However, current treatments often suffer from a user's inability to effectively adhere to and maintain continuous utilization of the therapeutic.

It would be desirable, therefore, to provide for personalization and tailoring of feedback-based interventions in the medical field that reduce patient risk, monitor for disease indicators, assess and analyze risk levels, determine tolerance, and provide for recommendations and treatment plans.

It would be further desirable to provide digital therapeutic treatments that provide data-driven and personalized real-time responsiveness, resulting in possible adjustment of treatment regimens based on iterative factors in a remote computing environment.

It would be yet further desirable to provide a recommendation and personalization engine for increasing yield and efficiency of a digital therapeutic user interaction, efficacy, and adherence in a remote computing environment.

It would be yet further desirable to mimic/automate recommendation and personalization features provided by a human, without the need for human input or intervention.

Thus, provided herein are apparatuses, systems and methods for such features.

### BRIEF DESCRIPTION OF THE DRAWINGS

FIG. 1 illustrates a block diagram of a distributed computer system that can implement one or more aspects of an embodiment of the present invention;

FIG. 2 illustrates a block diagram of an electronic device that can implement one or more aspects of an embodiment of the invention;

FIGS. 3A-5B illustrate one or more aspects of an embodiment of the invention;

FIGS. 6-7 illustrate processes that can implement one or more aspects of an embodiment of the invention.

While the invention is described with reference to the above drawings, the drawings are intended to be illustrative, and the invention contemplates other embodiments within the spirit of the invention

### DETAILED DESCRIPTION

The present invention will now be described more fully hereinafter with reference to the accompanying drawings which show, by way of illustration, specific embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as devices or methods. Accordingly, the present invention may take the form of an entirely hardware embodiment, an entirely software embodiment, or an embodiment combining software and hardware aspects. The following detailed description is, therefore, not to be taken in a limiting sense.

Throughout the specification and claims, the following terms take the meanings explicitly associated herein, unless the context clearly dictates otherwise. The phrases “in one embodiment,” “in an embodiment,” and the like, as used herein, does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” includes plural references. The meaning of “in” includes “in” and “on.”

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

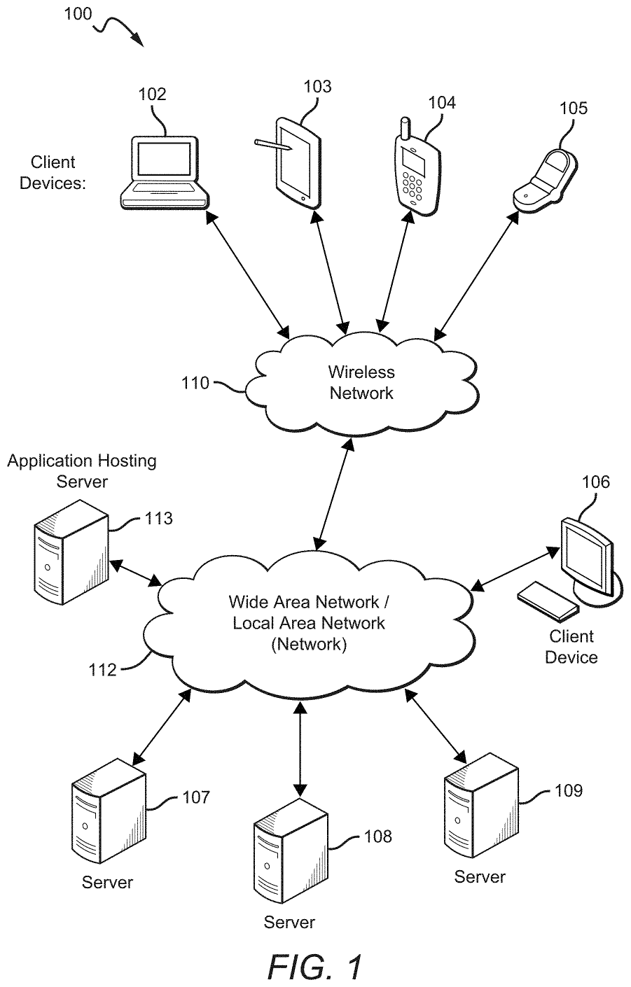

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. The servers 107-109 can include, for example, one or more application servers, content servers, search servers, and the like. FIG. 1 also illustrates application hosting server 113.

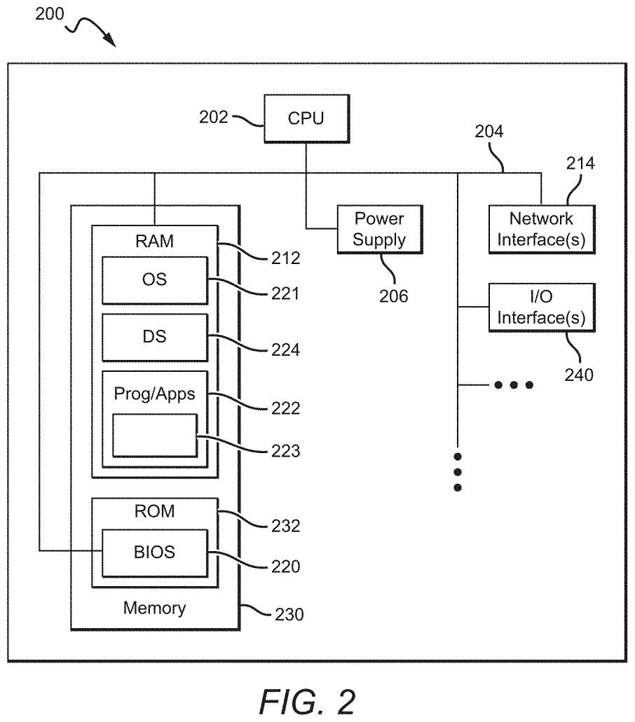

FIG. 2 illustrates a block diagram of an electronic device 200 that can implement one or more aspects of the assessment and recommendation engine for increasing yield in a remote computing environment (the “Engine”) according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the Engine program 223. The ROM 232 can also include Basic Input/Output System (BIOS) 220 of the electronic device.

Software aspects of the Engine program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components, and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Engine. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the above may be implemented in the Engine program 223. The Engine program 223 may be implemented on one or more client devices 102-106, one or more servers 107-109, and 113, or a combination of one or more client devices 102-106, and one or more servers 107-109 and 113.

Described herein is a recommendation and personalization engine for increasing yield and efficiency on a remote computing device.

In an embodiment, the engine minimizes risk, and increases adherence to requirements of the remote computing system.

In another embodiment, the engine increases efficiency and yield of a digital therapeutic or process, by, for example, maintaining efficacy, user interaction or adherence in a remote computing environment.

In an additional embodiment, the engine provides for personalization and tailoring of feedback-based interventions in the medical field, reducing risk and providing indication monitoring. The engine may further provide alarms and alerts for risk levels and remediate any inefficiencies.

In yet another embodiment, the engine may also provide for personalized recommendation and treatment plans, utilizing iterative factors and real-time responsiveness to adjust treatment regimens in the remote environment.

Thus, in accordance with an embodiment, provided is a personalized, repeated, forced-choice preference assessment system for the establishment of reinforcement algorithms. Such a system provides methods for increasing adherence and reducing risk in a digital remote environment.

The system may implement the following steps, in order to identify, personalize and re-distribute preference hierarchies of stimuli in the remote computing environment:
(1) Establishing a preference hierarchy, including identifying stimuli as being preferred, non-preferred, and highly preferred;
(2) Using stimuli identified as highly preferred to be used as reinforcers; and
(3) Using the stimuli as reinforcers, providing increased yield and efficiency.

The system assesses a forced-choice preference in the remote environment. This addresses the problem inherent in remote computing systems for digital therapeutic treatments of replicating the efficacy and yield, and therefore personalized treatment, of a doctor, without the involvement of a doctor.

The system may identify overall preference hierarchies for stimuli specifically for the remote computing environment, using a series of assessments. A preference hierarchy may then be established. The system may identify reinforcers for the environment, tiering stimuli into non-preferred, preferred and highly preferred stimuli for use as reinforcers. It should be noted that the stimuli may be tiered into any other suitable tiers.

In an embodiment, stimuli identified as, for example, highly preferred, may be used as reinforcers. The reinforcers may be delivered to a user in the remote environment contingent on the occurrence of a trigger. For example, a trigger may include a targeted behavior, or input in the remote environment. In a further example, reinforcers may be delivered upon occurrence of a predetermined threshold number of triggers, such as, for example, two or three behaviors.

The system repeatedly and iteratively identifies a preference hierarchy for environmental stimuli in the environment, through a forced-choice preference assessment. Information determined in the assessment is then used to present environmental stimuli as reinforcers to increase yield and efficiency in future tasks. In an embodiment, the reinforcers may be presented in a paired forced-choice paradigm, or any other suitable form.

The systems and methods disclosed herein may be used to increase a user's yield in the environment. This may increase treatment yields and efficacies in a digital therapeutic product by, for example, creating accord between the digital therapeutic and the user in the assignment of tasks in the environment and facilitating more efficient and accurate achievement of goals in the remote environment.

For example, the systems and methods provide a choice paradigm, creating a working alliance hierarchy to provide repeated choices for treatment. In doing so, users are presented with a series of opportunities, increasing system and user autonomy and efficacy, thereby resulting in increased yield of the digital therapeutic.

The preference assessment may be utilized to maximize efficacy and reduce error of a reinforcer in the remote environment. In another embodiment, the following steps may be implemented as part of a preference assessment.

In a first step, the preference assessment creates a preference hierarchy, which is a set of digital dependencies used to determine which resulting sequences may be most reinforcing to a particular individual at a given time. In a second step, the reinforcer may be used once a trigger occurs, thereby redirecting a sequence in a second direction, to increase yield and efficiency. It should be noted that, as in most remote computing environments, preference changes occur frequently and rapidly, resulting in fluctuating states of preference assessment. Thus, the invention preferably iteratively changes over time, based on the fluctuations in preference assessment, and results in iterative and personalized changes in preference assessment to remediate any trigger changes.

The preference assessment, in accordance with an embodiment, may be presented as a paired-stimulus ciphering in the remote computing environment. The preference assessment may be presented as two simultaneous stimuli. That is, one, two or more stimuli may be selected from an array of stimuli.

In one step, a user may be queried to indicate a stimulus hierarchy or preference out of two or multiple presented stimuli. After a first query, second and subsequent queries are presented. In an embodiment, a user is queried with two different stimuli, in unique pairings. The queries may then be repeated until all possible pairs of stimuli have been presented.

Therefore, since stimuli are presented in all possible pairings, a preferential hierarchy is established. This allows for a more individual-focused digital therapeutic remote environment, increasing efficacy and thereby reducing erroneous digital therapeutic treatments.

In one embodiment, the system preferably provides reinforcing consequential results, by providing a consistent choice between two stimuli in the preferential hierarchy. For example, a user may be prompted with “please select whether you wish to complete a mission involving (1) a support network; or (2) a reward badge.” By selecting one of the provided stimuli, the user completes a selection, instead of ignoring the process, and therefore increases yield and efficacy of the digital therapeutic.

In an example, the invention therefore provides determination of a user preference for reinforcers, and allows for creating the preferential hierarchy. This increases the user's involvement in the remote environment, and improves digital therapeutic outcomes by personalizing outputs to reflect user preferences.

### Establishing a Preference Hierarchy

A preference hierarchy may be established using an inverse preferential hierarchy. As a first step, an initial analysis is performed by calculating a baseline inverse hierarchy based on an initial assessment. At a second step, the inverse hierarchy is updated based on sequential force choice prompts (e.g., the stimuli pairs). At a third step, the inverse hierarchy is tracked. At a fourth step, feedback is provided on latency of choice selection. This ensures continuous user personalization and adherence.

In one embodiment, four preference groups (PGs) are provided: Attention (PGA), Activity (PGP), Independent Rewards (PGI) and Tangible (PGT). An initial determination is performed, in order to calculate the inverse hierarchy, using the following equation:

$$C ( n , r ) = \frac{n !}{( r ! ( n - r ) ! )}$$

In accordance with this equation, n is the set or population, and r is the subset of n. It should be noted that, in the current embodiment, four preference groups are utilized, though any suitable number of preference groups are contemplated by the invention.

Referring back to the embodiment, with four preference groups, n=4. Each query provides a choice pair of two of the preference groups at a time, with r=2. Therefore, six total combinations are possible, with the user being prompted for each of these six combinations a total of four times. Therefore, twenty-four total choice pairs are provided.

Once the total number of choice pairs are provided, a preference ratio (PR) is calculated for each PG. The ratio is calculated with the following equation:

$$PR = \frac{Number of PG selected}{Total Number of Choice Pairs}$$

Each Preference Group therefore has a Preference Ratio ranging from 0 to 1. With a PR of 0, the user indicates a predictability of never selecting the specific PG when given an option, whereas, at the other extreme, with a PR of 1, the user indicates a predictability of always selecting the specific PG, when given the option.

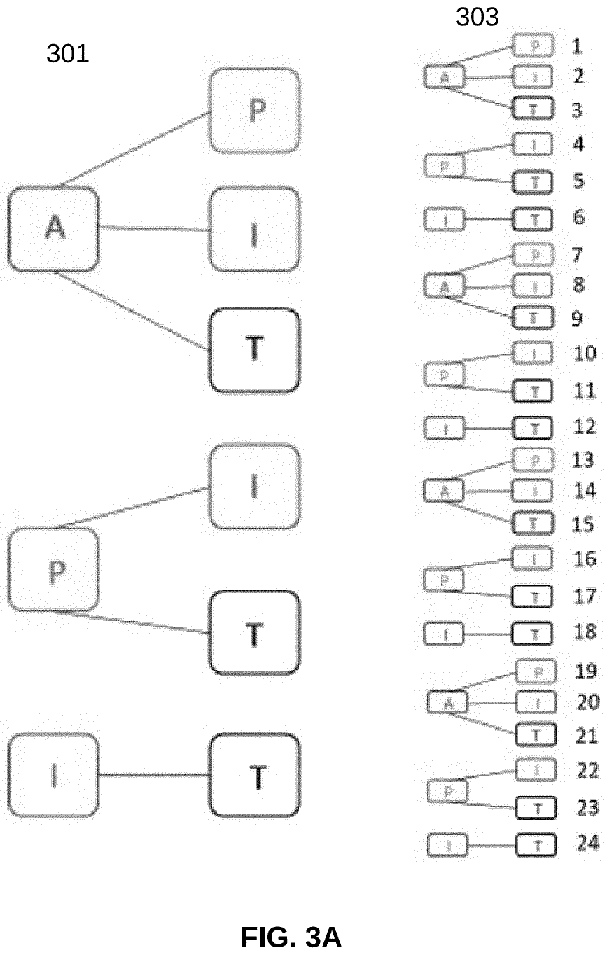

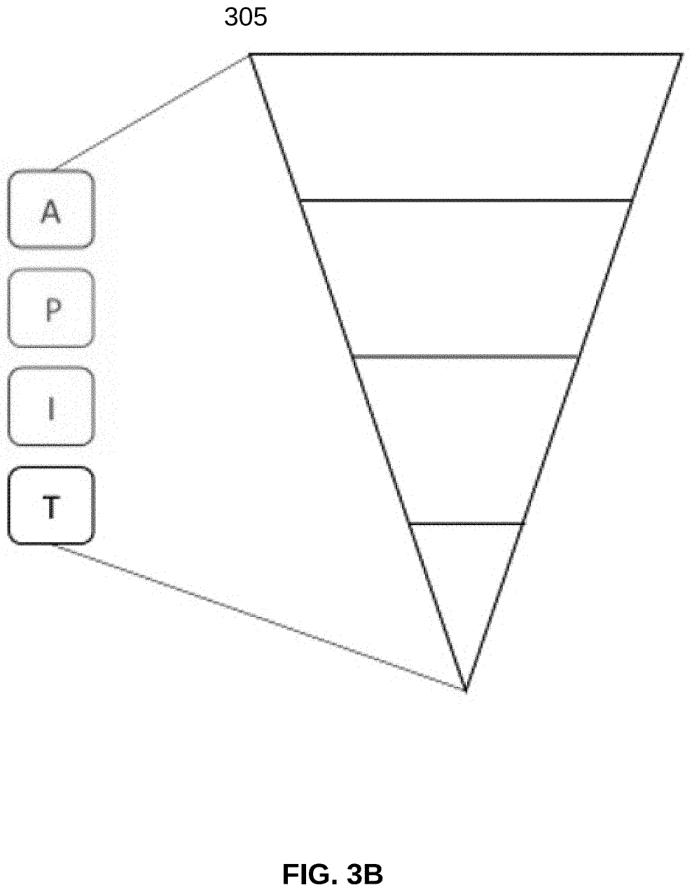

FIGS. 3A-3B are an illustration of the various assessments. 301 illustrates various Preference Groups and their preferred stimulus. 303 illustrates this in more detail, with 305 illustrating the inverse PG hierarchy.

TABLE 1

1. PR of PG_{T} = 1.0
2. PR of PG_{A} = 0.67
3. PR of PG_{P} = 0.33
4. PR of PG_{I} = 0

Table 1, shown above, illustrates a range of Preference Ratios for the various Preference Groups.

In a further example, if a user selects PG_{T} 12 times when presented with 12 choice pairs that included PG_{T} (For example—PG_{T} vs. PG_{P}, PG_{T} vs. PG_{I} and PG_{T} vs. PG_{A}), PG_{A} 8 times when presented with 12 choice pairs that included PG_{A} (For example—PG_{A} vs. PG_{I}, PG_{A} vs. PG_{P} and PG_{T} vs. PG_{A}), PG_{P} 4 times when presented with 12 choice pairs that included PG_{P} (For example—PG_{T} vs. PG_{P}, PG_{A} vs. PG_{P} and PG_{P} vs. PG_{I}) and PG_{I} 0 times when presented with 12 choice pairs that included PG_{I} (For example—PG_{T} vs. PG_{I}, PG_{A} vs. PG_{I} and PG_{P} vs. PG_{I}) the baseline PRs and inverse preference hierarchy would be that as shown above in Table 1.

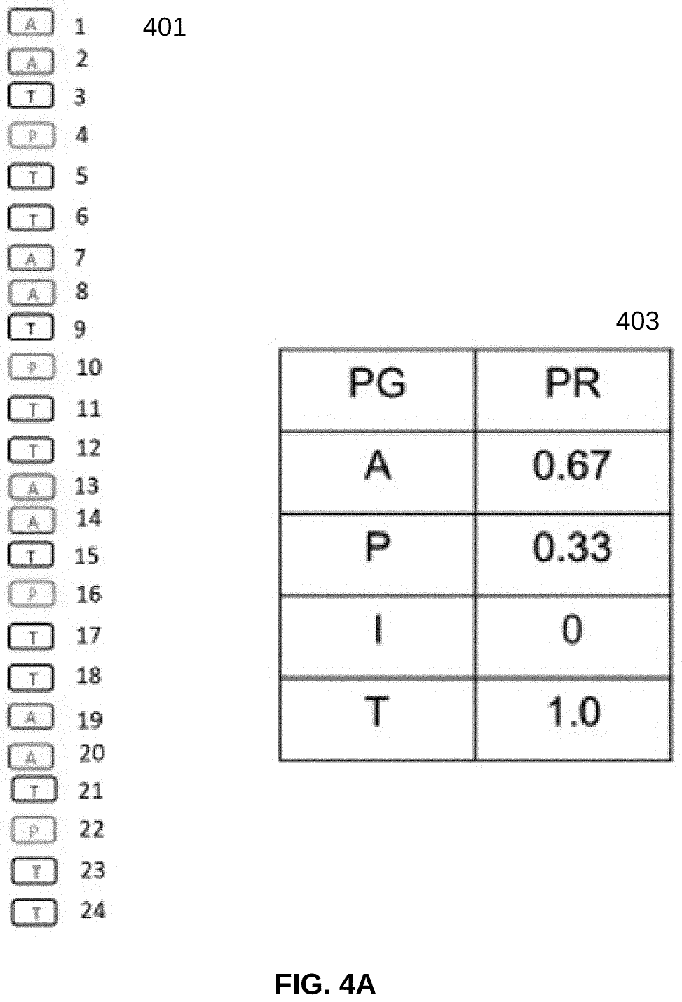

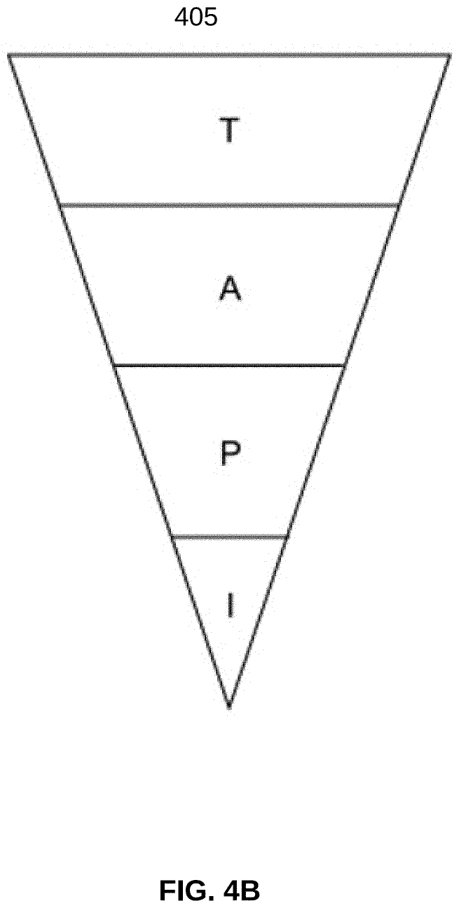

As shown in FIGS. 4A-4B, 401 is a visual representation of this, illustrating the preferred stimulus in each of 24 presented pairs. 403 is a table showing the Preference Ratios for each Preference Group. 405 illustrates the inverse preference hierarchy, with the most preferred PG being at the top of the inverted pyramid.

At the next step, the user may begin treatment. Specifically, users may be presented with a plurality of queries, each with choice pairs of reinforcing consequences. Each query may be categorized into one of the 4 PGs disclosed.

At a next step, each time a consequence is selected, a response is recorded. The inverse preference hierarchy is then updated and personalized to reflect the newly calculated Preference Ratio for each PG.

The following query may then only be triggered based on higher Preference Ratios, reflecting the user's preference. Thus, subsequent choice pairs presented may only be those with a PR above a threshold level (such as, for example, 0.5) or a those in the top two PRs.

Following this, during treatments, a user may be periodically shown choice pairs including consequences in PGs that were not in their top PR. This ensures continuous and non-biased updating of the inverse hierarchy. Such choice pairs may be shown at preset interval periods, such as daily, weekly, or monthly, or upon completion of a predetermined number of treatments. In a further example, choice pairs may be shown in PGs that are not in the top PRs upon a determination that a user may not be adhering to treatment, or may not be participating effectively or accurately in the digital therapeutic treatments. For example, a user may be proceeding with digital therapeutic at a quicker rate than acceptable, at which point the system may flag the user's behavior as consistent with reduced yield and efficacy. Accordingly, the user may then be presented with additional choice pairs to increase yield, including consequences in PGs not in the top PR.

TABLE 2

4. PR of PG_{I} = 0
3. PR of PG_{P} = 0.33
2. PR of PG_{A} = 0.67
1. PR of PG_{T} = 1

In a further example, a user may have the baseline PRs and inverse preference hierarchy shown above in Table 2. The user may subsequently undergo one week of treatment, creating one therapeutic mission daily. During the one week of treatment, the user may have selected the following choice pair prompts:
{PG_{A}, (PG_{T} vs. PG_{A})}, {PG_{P}, (PG_{T} vs. PG_{P})}, {PG_{A}, (PG_{T} vs. PG_{A})}, {PG_{A}, (PG_{A} vs. PG_{I})}, {PG_{I}, (PG_{P} vs. PG_{I})}, {PG_{A}, (PG_{A} vs. PG_{P})}, {PG_{A}, (PG_{T} vs. PG_{A})}.

Based on the selection, the user's updated Preference Ratios and inverse preference hierarchy at the end of the first week of treatment would be as shown in Table 3, below:

TABLE 3

1. PR of PG_{I} = 0.07,
2. PR of PG_{P} = 0.30
3. PR of PG_{A} = 0.76
4. PR of PG_{T} = 0.75

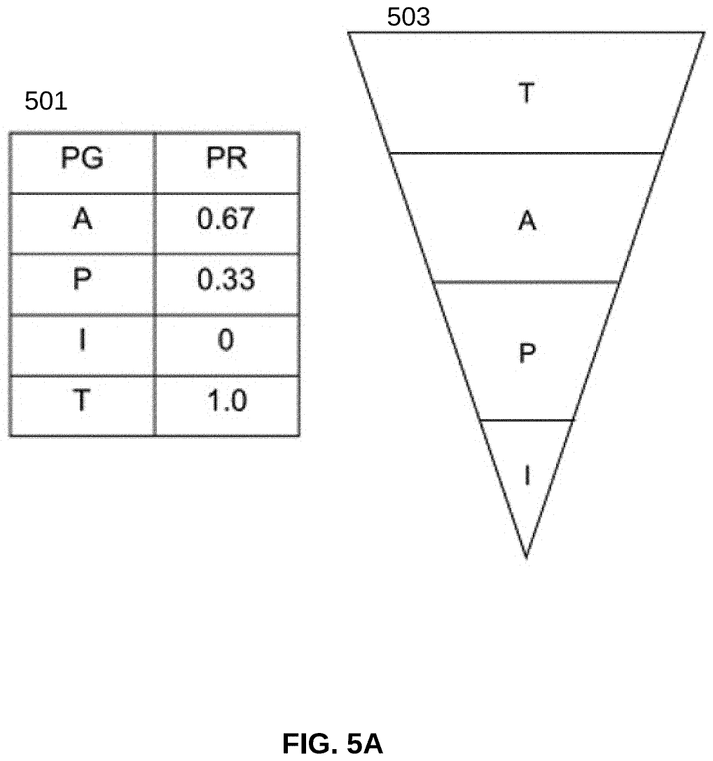

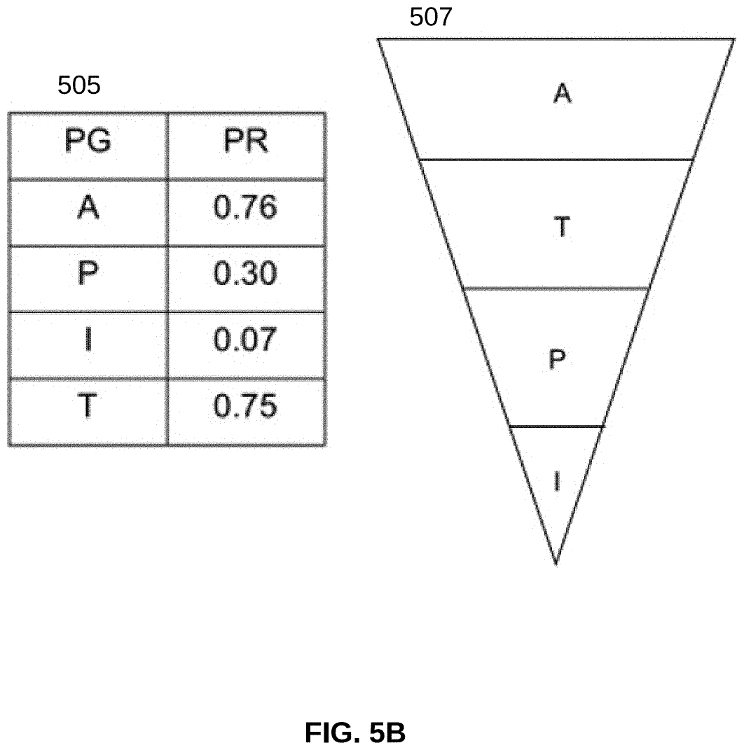

FIGS. 5A-5B illustrate a visual representation of the features of Table 3.

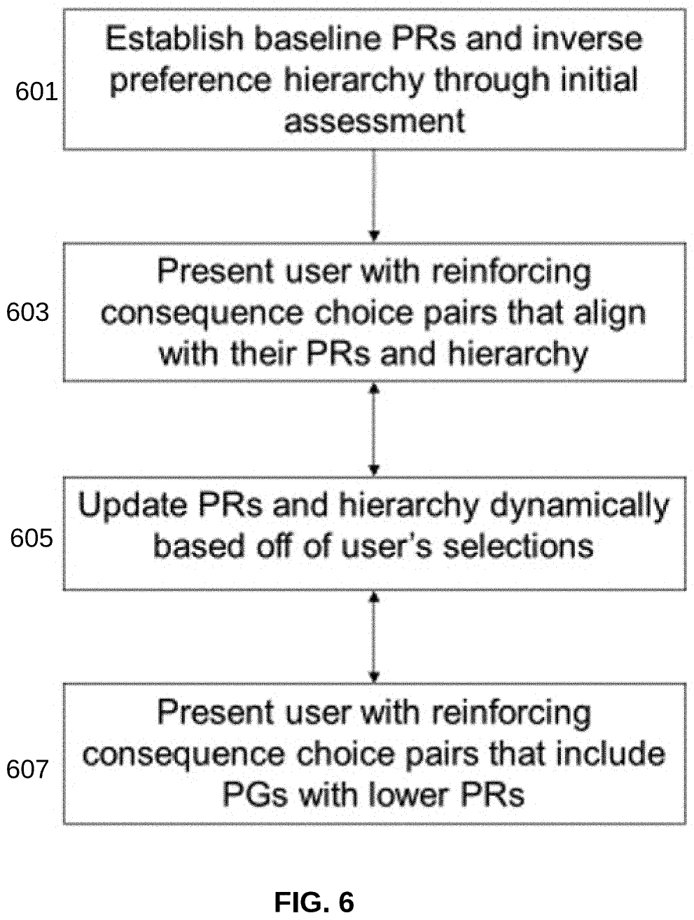

FIG. 6, illustrates is a process, in accordance with an embodiment. At a first step, in step 601, baseline Preference Ratios are established. An inverse preference hierarchy is also established, through initial assessment. In step 603, a user is then presented with reinforcing consequence choice pairs, which align with the PRs and hierarchy. In step 605, PRs and the hierarchy are then iteratively and dynamically updated based on the user's selections. In step 607, the user is then presented with reinforcing consequence choice pairs, including PGs with lower PRs.

In an example, a user's click speed may be measured in an application. The click speed data may then be utilized and measured against predetermined acceptable and non-acceptable speeds. The system may then calculate the speed at which a user is deciding between reinforcing consequences choice pairs, referred to as click decision speed (CDS). CDS is defined as a change in time:

CDS=Δt=t_{S}−t_{A}

Therefore, t_{S} is the time at which the user makes a selection between two choice pairs, and t_{A} is the time at which the choice pairs first appear on the screen.

For example, during the first 2 weeks of treatment, CDS may be calculated to find a baseline for a user. In this embodiment, CDS utilizes responsive time as the variable. However, other variables, such as time of day, can also be tracked, as users may exhibit differences in CDS depending on time of day. Additional variables may include, but are not limited to, gender, age, total usage of mobile device on that day (for example, receiving such information from the mobile device), pupil dilation determined via a camera of a mobile device, or any other suitable indicator. For example, the system may instruct a camera on the device to measure/assess pupil dilation, and provide the results.

In the example above, a user may be associated with three CDS values: CDS_{M} (Morning CDS or CDS within the hours of 5 AM to 11:59 AM, inclusive), CDS_{A} (Afternoon CDS or CDS within the hours of 12 PM to 5:59 PM, inclusive) and CDS_{N} (Night CDS or CDS within the hours of 6 PM to 4:59 AM, inclusive). Additional CDS values may be provided, based on the variables utilized.

After a baseline CDS is recorded, comparisons between CDS and baseline CDS for the time of day may be made each time a user is prompted with a reinforcing consequence choice pair and makes a selection. Accordingly, as choice pairs and prompts are kept around the same length (up to 12 words in length), it is assumed that average reading speed is accounted for in CDS.

In other instances, where variables aside from responsive time is tracked, the process remains the same. A user is associated with one or more CDS values, corresponding to a baseline for that variable activity. After the baseline for the activity is recorded, a comparison between the current CDS and baseline CDS occurs.

Returning to the responsive time embodiment, users may be allowed a deviation, such as, for example, up to 1 second of difference from baseline CDS before the selection was recorded as a deviation. It should be noted that this deviation is merely an example, and any other suitable deviation may be utilized.

In a further example, after a preset number of deviations, such as three, have been recorded for a user, the user may trigger a warning. The warning may indicate that the user has exceeded allowable time, or is not proceeding with in the allowable time range, to make a decision for their treatment within the past three days (or other suitable time frame). In an embodiment, this trigger/result may be stored in a database either on the user's mobile device or a server communicatively coupled to the mobile device.

The user may then receive personalized messages, encouraging them to continue treatment and personalizing it to their preferences.

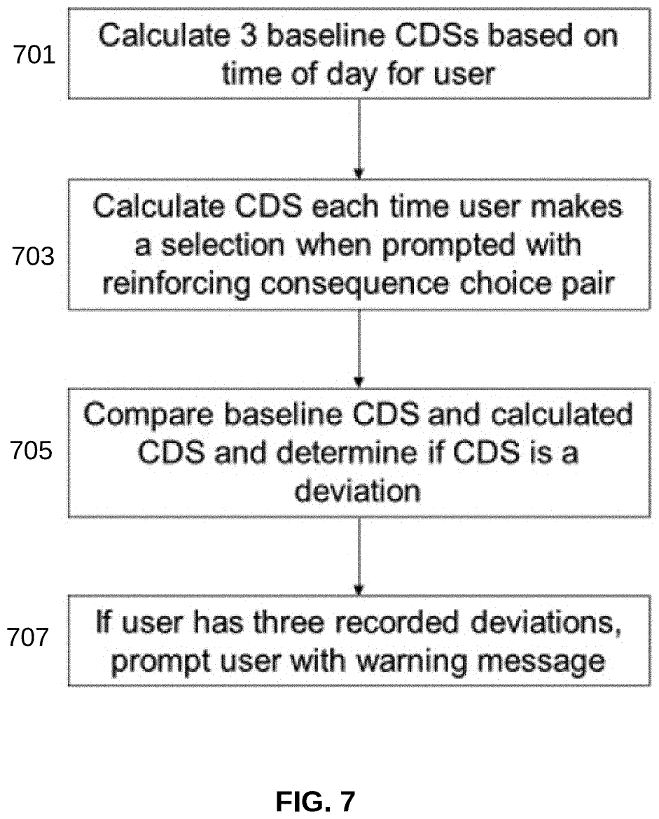

FIG. 7 illustrates a process for assessing click speed data, sometimes referred to as click decision data (CDS). The following may also be referred to as a click decision data assessment system. In a first step, step 701, three baseline CDS are calculated, each for a different time of day, for each user. The different times of day may be no less than 4 hours apart. In a second step, step 703, each time a user makes a selection when prompted with a reinforcing consequence choice pair (RCCP), a CDS is calculated. In a third step, step 705, the baseline CDS is compared to a calculated CDS, to determine if the calculated CDS presents a deviation. At a fourth step, step 707, if the user presents more than a predetermined allowable number of recorded deviations, the user is prompted with a warning message or other suitable result. The warning message or other suitable result may be displayed on or more user devices. For example, it may displayed on a smartphone and/or a smart watch.

A high-level process, in accordance with an embodiment, may include the following: (1) Providing a preference hierarchy (for example, 4 tiers); (2) Ranking the hierarchy from most to least preferred; (3) Resulting in a likely change of reinforcer preference over time, due to fluctuating user efficacy rates, resulting from non-adherence; (4) Regularly re-assessing user preference hierarchy (at regular intervals, either based on milestones and/or time); (5) Utilizing this efficacy and yield increase to complete a digital therapeutic treatment; (6) Providing user choice and autonomy to select reinforcing consequence; (7) Utilizing user selection to maintain similar therapeutic content delivered to all users, but varying the order and manner of presentation in customization to a user's preference.

It should be noted that multiple Preference Groups may be in the same tier, due to an identical equal preference.

Further, the invention, as disclosed herein, addresses a problem unique to technology-replicating yield, efficacy and bond of two individuals in a digital remote environment, when only one individual is present.

In an embodiment, reinforcers selected more frequently may be presented to the user more frequently, and vice versa.

Pseudocode, in accordance with an embodiment of the present invention, is below. In the pseudocode, CTA is a call-to-action, a CTA selection is a call-to-action item that a user selections in a lesson, and a CTA bucket is the predefined concept that a call-to-action item is associated with (such as “Work & Study”, “Get 5 minutes of exercise” and the like).

process to find preferred CTA buckets for a user:

pull user's all CTA choices selected in all completed lessons
including repeated ones
initialize CTA bucket weights as bucket_values = {

‘Physical health & wellness’: 0,
‘Social connections’: 0,
‘Self care & development’: 0,
‘Work & study’: 0

}
for each CTA choice

find related CTA bucket b and weight w
increase bucket_values[b] by w

initialize preferred_buckets = [ ]
for each CTA bucket b:

if bucket_values[b] >= 2 and bucket_values[b] >= 120% *
average(bucket_values):

add bucket b to preferred_buckets

return preferred_buckets

According to the above pseudocode, N of 1 personalization finds a user's preferred topic, and sends messages (such as SMS or app alerts) related to those preferred topics.

While this invention has been described in conjunction with the embodiments outlined above, many alternatives, modifications and variations will be apparent to those skilled in the art upon reading the foregoing disclosure. Accordingly, the illustrative embodiments of the invention, as set forth above, are intended to be illustrative, not limiting. Various changes may be made without departing from the spirit and scope of the invention.

What is claimed is:

1. A computer system for assessing click decision data in a remote computing environment comprising one or more processors, one or more computer-readable memories, and one or more computer-readable storage devices, and program instructions stored on at least one of the one or more storage devices for execution by at least one of the one or more processors via at least one of the one or more memories, the stored program instructions comprising:
determining a plurality of baseline click decision data (CDS) values for a user;
following the determining of the plurality of baseline CDS values for the user, determining a subsequent CDS value each time a user makes a selection when prompted with a prompt;
for each of the subsequent CDS values, comparing the subsequent CDS value to the plurality of baseline CDS values;
determining whether the subsequent CDS value presents a predetermined deviation from the plurality of baseline CDS values;
incrementing a recorded deviations for each of the predetermined deviation;
if the recorded deviations exceeds a predetermined allowable number of deviations, present, on one or more screens of a user device, a warning message to the user.

2. The click decision data assessment system according to claim 1, wherein the prompt is a reinforcing consequence choice pair (RCCP).

3. The click decision data assessment system according to claim 1, wherein each of the plurality of baseline CDS values for the user is determined for a plurality of different times of day.

4. The click decision data assessment system according to claim 3, wherein the plurality of different times of day is morning, afternoon, evening, and night.

5. The click decision data assessment system according to claim 3, wherein the plurality of different times of day comprises 7 AM, noon, 3 PM, 5 PM, 10 PM, and midnight.

6. The click decision data assessment system according to claim 3, wherein the plurality of different times of day comprises times that are no less than 4 hours apart.

7. The click decision data assessment system according to claim 1, wherein determining a plurality of baseline CDS values comprises measuring the user's click speed in response to a first prompt in an application running on a mobile device.

8. The click decision data assessment system according to claim 7, wherein measuring the user's click speed in response to a first prompt in an application running on a mobile device comprises determining a time between a display of the first prompt and a user touching a first area of a touchscreen of the one or more screens of the user device.

9. The click decision data assessment system according to claim 1, wherein comparing the subsequent CDS to the plurality of baseline CDS values comprises determining a first time of day of the subsequent CDS, determining an average CDS value of a subset of the plurality of baseline CDS values corresponding to times closest to the first time of day, and determining a difference between the subsequent CDS and the subset.

10. A computer implemented method for click decision data assessment, the method comprising:
determining a plurality of baseline click decision data (CDS) values for a user;
following the determining of the plurality of baseline CDS values for the user, determining a subsequent CDS value each time a user makes a selection when prompted with a prompt;
for each of the subsequent CDS values, comparing the subsequent CDS value to the plurality of baseline CDS values;
determining whether the subsequent CDS value presents a predetermined deviation from the plurality of baseline CDS values;
incrementing a recorded deviations for each of the predetermined deviation;
if the recorded deviations exceeds a predetermined allowable number of deviations, present, on one or more screens of a user device, a warning message to the user.

11. The click decision data assessment system according to claim 1, wherein the prompt is a reinforcing consequence choice pair (RCCP).

12. The click decision data assessment system according to claim 1, wherein each of the plurality of baseline CDS values for the user is determined for a plurality of different times of day.

13. The click decision data assessment system according to claim 3, wherein the plurality of different times of day is morning, afternoon, evening, and night.

14. The click decision data assessment system according to claim 3, wherein the plurality of different times of day comprises 7 AM, noon, 3 PM, 5 PM, 10 PM, and midnight.

15. The click decision data assessment system according to claim 3, wherein the plurality of different times of day comprises times that are no less than 4 hours apart.

16. The click decision data assessment system according to claim 1, wherein determining a plurality of baseline CDS values comprises measuring the user's click speed in response to a first prompt in an application running on a mobile device.

17. The click decision data assessment system according to claim 7, wherein measuring the user's click speed in response to a first prompt in an application running on a mobile device comprises determining a time between a display of the first prompt and a user touching a first area of a touchscreen of the one or more screens of the user device.

18. The click decision data assessment system according to claim 1, wherein comparing the subsequent CDS to the plurality of baseline CDS values comprises determining a first time of day of the subsequent CDS, determining an average CDS value of a subset of the plurality of baseline CDS values corresponding to times closest to the first time of day, and determining a difference between the subsequent CDS and the subset.
